(19) United States
(12) Patent Application Publication
(10) Pub. No.: US 2026/0174725 A1
(43) Pub. Date: Jun. 25, 2026

(54) SOLID DISPERSION AGENT OF DIMYRICETIN-YL-DISELENIDE AND APPLICATION THEREOF

(71) Applicant: Shanghai Aiqi Pharmaceutical Co., Ltd., Shanghai (CN)

(72) Inventors: Kunyuan Song, Shanghai (CN); Weiwei Chen, Shanghai (CN)

(21) Appl. No.: 19/001,490

(22) Filed: Dec. 25, 2024

(51) Int. Cl. A61K 31/352; A61K 47/38; A61K 47/58

(52) U.S. Cl. CPC A61K 31/352; A61K 47/38; A61K 47/58

(57) ABSTRACT

A solid dispersion agent of dimyricetin-yl-diselenide and an application thereof are provided. The solid dispersion agent of dimyricetin-yl-diselenide includes dimyricetin-yl-diselenide and hydroxypropyl methylcellulose acetate succinate MG, with a mass ratio of 1:3-5. The solid dispersion agent of dimyricetin-yl-diselenide includes dimyricetin-yl-diselenide and polyvinylpyrrolidone K30, with a mass ratio of 1:3-5. The present disclosure further discloses an application of hydroxypropyl methylcellulose acetate succinate MG as a solid dispersion agent of dimyricetin-yl-diselenide. The present disclosure further discloses an application of polyvinylpyrrolidone K30 as the solid dispersion agent of dimyricetin-yl-diselenide. The dimyricetin-yl-diselenide in the present disclosure can be effectively dissolved, with an optimal concentration of 0.7 mg/mL, thereby improving the bioavailability of dimyricetin-yl-diselenide in pharmaceutical applications.

### TECHNICAL FIELD

The present disclosure relates to the field of medicine technologies, and in particular, to a solid dispersion agent of dimyricetin-yl-diselenide and an application thereof.

### BACKGROUND

Dimyricetin-yl-diselenide (DMS) has extremely high medicinal value. For example, Chinese patent No. 111450088B discloses that dimyricetin-yl-diselenide has therapeutic effects on tumors and novel coronavirus and has obvious inhibitory effects on seven different human tumor cells. Its IC50 value for the target site Mpro of novel coronavirus (2019-nCOV) is 0.807±0.0830 μM, which has significant effects.

However, dimyricetin-yl-diselenide is difficult to dissolve in water, and is extremely soluble in methanol, ethanol, propylene glycol, glycerol, etc. It is slightly soluble in Tween 80, Capriol 90, etc., slightly soluble in Capmul MCM, Labrasol, etc., and soluble in NMP, PEG400, PEG200, DMSO, etc. Due to its insoluble nature, the bioavailability of dimyricetin-yl-diselenide has been somewhat affected in pharmaceutical applications.

### SUMMARY

The present disclosure aims to improve problems in the prior art by providing a solid dispersion agent of dimyricetin-yl-diselenide and an application thereof so as to enhance its bioavailability in pharmaceutical applications.

A solid dispersion agent of dimyricetin-yl-diselenide, including dimyricetin-yl-diselenide and hydroxypropyl methylcellulose acetate succinate MG,
where a mass ratio of dimyricetin-yl-diselenide to hydroxypropyl methylcellulose acetate succinate MG is 1:3-5.

In some embodiments of the present disclosure, the mass ratio of dimyricetin-yl-diselenide to hydroxypropyl methylcellulose acetate succinate MG is 1:4.

A solid dispersion agent of dimyricetin-yl-diselenide, including dimyricetin-yl-diselenide and polyvinylpyrrolidone K30,
where a mass ratio of dimyricetin-yl-diselenide to polyvinylpyrrolidone K30 is 1:3-5.

In some embodiments of the present disclosure, the mass ratio of dimyricetin-yl-diselenide to polyvinylpyrrolidone K30 is 1:4.

An application of hydroxypropyl methylcellulose acetate succinate MG as a solid dispersion agent of dimyricetin-yl-diselenide.

In some embodiments of the present disclosure, in the above application, a mass ratio of dimyricetin-yl-diselenide to hydroxypropyl methylcellulose acetate succinate MG is 1:3-5.

In some embodiments of the present disclosure, the mass ratio of dimyricetin-yl-diselenide to hydroxypropyl methylcellulose acetate succinate MG is 1:3-5.

An application of polyvinylpyrrolidone K30 as a solid dispersion agent of dimyricetin-yl-diselenide.

In some embodiments of the present disclosure, a mass ratio of dimyricetin-yl-diselenide to polyvinylpyrrolidone K30 is 1:3-5.

In some embodiments of the present disclosure, in the above application, the mass ratio of dimyricetin-yl-diselenide to polyvinylpyrrolidone K30 is 1:3-5.

Beneficial effects: the solid dispersion agent of dimyricetin-yl-diselenide and the application thereof disclosed in the present disclosure have the following beneficial effects.

The dimyricetin-yl-diselenide can be effectively dissolved, with an optimal concentration reaching 0.7 mg/mL, which improves the bioavailability of dimyricetin-yl-diselenide in pharmaceutical applications.

### BRIEF DESCRIPTION OF DRAWINGS

FIG. 1 shows a schematic diagram of tumor volume measurement results for different mice in Test Example 2.

FIG. 2 is a schematic diagram of tumor growth inhibition rate in different 5 experimental groups of mice in Test Example 2.

### DESCRIPTION OF EMBODIMENTS

The specific embodiments of the present disclosure will be described in detail below.

In the present application, a concentration analysis method is as follows table 1.

TABLE 1

| High performance liquid | Agilent 1200 HPLC(GP-FRD-LC-06) | | |
|---|---|---|---|
| chromatograph (HPLC) | | | |
| Mobile phase | A: 0.1% methanoic acid - aqueous solution | | |
| | B: 0.1% methanoic acid - acetonitrile | | |
| | solution | | |
| | Time (min) | A % | B % |
| Gradient elution program | 0 | 95 | 5 |
| | 3 | 50 | 50 |
| | 7 | 5 | 95 |
| | 10 | 5 | 95 |
| | 10.1 | 95 | 5 |
| | 15 | 95 | 5 |

| | |
|---|---|
| Chromatographic column | Waters SunFire ™ C18 (4.6 mm * |
| | 150 mm, 3.5 μm) |
| Column temperature (° C.) | 25 |
| Detector | DAD |
| Test wavelength (nm) | 254 |
| Sampling volume (μL) | 20 |
| Flow velocity (mL/min) | 1.0 |
| Run time (min) | 15 |

### Example 1

A solid dispersion agent of dimyricetin-yl-diselenide, including dimyricetin-yl-diselenide and hydroxypropyl methylcellulose acetate succinate MG.

A mass ratio of dimyricetin-yl-diselenide to hydroxypropyl methylcellulose acetate succinate MG is 1:3.

### Example 2

A solid dispersion agent of dimyricetin-yl-diselenide, including dimyricetin-yl-diselenide and hydroxypropyl methylcellulose acetate succinate MG.

A mass ratio of dimyricetin-yl-diselenide to hydroxypropyl methylcellulose acetate succinate MG is 1:5.

### Example 3

A solid dispersion agent of dimyricetin-yl-diselenide, including dimyricetin-yl-diselenide and hydroxypropyl methylcellulose acetate succinate MG.

A mass ratio of dimyricetin-yl-diselenide to hydroxypropyl methylcellulose acetate succinate MG is 1:4, with a concentration of 0.5 mg/mL.

### Example 4

A solid dispersion agent of dimyricetin-yl-diselenide, including dimyricetin-yl-diselenide and polyvinylpyrrolidone K30.

A mass ratio of dimyricetin-yl-diselenide to polyvinylpyrrolidone K30 is 1:3.

### Example 5

A solid dispersion agent of dimyricetin-yl-diselenide, including dimyricetin-yl-diselenide and polyvinylpyrrolidone K30.

A mass ratio of dimyricetin-yl-diselenide to polyvinylpyrrolidone K30 is 1:5.

### Example 6

A solid dispersion agent of dimyricetin-yl-diselenide, including dimyricetin-yl-diselenide and polyvinylpyrrolidone K30.

A mass ratio of dimyricetin-yl-diselenide to polyvinylpyrrolidone K30 is 1:4, with a concentration of 0.7 mg/mL.

### Performance Testing

### Comparison Example

Animal information: SD rats, 170-210 g, male
Administration way: oral gavage (administrating by gavage after dimyricetin-yl-diselenide being direct suspended in water)
Dosage: 100 mg/kg

The test results are as follows table 2.

TABLE 2
Individual and average plasma concentration/time data after oral administration of
100 mg/kg DMS in male SD rats

| | | | Concentration | | | | | |
|---|---|---|---|---|---|---|---|---|
| Dosage | Administration | Sampling | (ng/ml) | | | Average | | |
| (mg/Kg) | way | time (hr) | Rat #10 | Rat #11 | Rat #12 | (ng/ml) | SD | CV(%) |
| 100 | Oral | 0.25 | BQL | BQL | BQL | BQL | BQL | BQL |
| MW | administration | 1 | BQL | BQL | BQL | BQL | BQL | BQL |
| | 792.37 | 2 | BQL | BQL | BQL | BQL | BQL | BQL |
| | | 4 | BQL | BQL | BQL | BQL | BQL | BQL |
| | | 8 | 43.9 | 15.3 | 33.3 | 30.8 | 14.5 | 46.9 |

| | | | | | Average | | |
|---|---|---|---|---|---|---|---|
| PK parameter | Unit | Rat #10 | Rat #11 | Rat #12 | (ng/ml) | SD | CV(%) |
| Tₘₐₓ | hr | NA | NA | NA | NA | NA | NA |
| Cₘₐₓ | ng/ml | NA | NA | NA | NA | NA | NA |
| T_{1/2} | hr | NA | NA | NA | NA | NA | NA |
| AUC ₗₐₛₜ | hr*ng/ml | NA | NA | NA | NA | NA | NA |
| AUC _{INF} | hr*ng/ml | NA | NA | NA | NA | NA | NA |
| MRT _{INF} | hr | NA | NA | NA | NA | NA | NA |
| Rsq_adjuseted | NA | NA | NA | NA | NA | NA | NA |
| Regression Points | hr | NA | NA | NA | NA | NA | NA |
| AUC ₗₐₛₜ | hr*μM | NA | NA | NA | NA | NA | NA |
| AUC _{INF} | hr*μM | NA | NA | NA | NA | NA | NA |
| Cₘₐₓ | μM | NA | NA | NA | NA | NA | NA |

From the above test results, it can be seen that a target compound was not detected in the plasma after dimyricetin-yl-diselenide was directly suspended in water and administered orally, and its bioavailability was basically 0.

### Test Example 1

Animal information: SD rats, 170-210 g, male
Administration way: oral gavage (mass ratio of dimyricetin-yl-diselenide to polyvinylpyrrolidone K30 being 1:4)
Dosage: 100 mg/kg

The results are as follows table 3.

TABLE 3
Individual and average plasma concentration/time data after oral administration of
100 mg/kg DMS in male SD rats

| | | | Concentration | | | | | |
|---|---|---|---|---|---|---|---|---|
| Dosage | Administration | Sampling | (ng/ml) | | | Average | | |
| (mg/Kg) | way | time (hr) | Rat #1 | Rat #2 | Rat #3 | (ng/ml) | SD | CV(%) |
| 500 | Oral | 1 | BQL | BQL | BQL | BQL | NA | NA |
| MW | administration | 4 | BQL | BQL | BQL | BQL | NA | NA |
| | 792.37 | 6 | 298 | 86.9 | 169 | 185 | 106 | 57.6 |
| | | 8 | 52.0 | 80.2 | 136 | 89.4 | 42.7 | 47.8 |
| | | 12 | 73.2 | 291 | 91.3 | 152 | 121 | 79.6 |
| | | 24 | 44.3 | 95.3 | 21.1 | 53.6 | 38.0 | 70.9 |
| | | 36 | BQL | BQL | BQL | BQL | NA | NA |
| | | | | | | Average | | |

| PK parameter | Unit | Rat #1 | Rat #2 | Rat #3 | (ng/ml) | SD | CV (%) |
|---|---|---|---|---|---|---|---|
| Tₘₐₓ | hr | 6.00 | 12.0 | 6.00 | 8.00 | 3.46 | 43.3 |
| Cₘₐₓ | ng/ml | 298 | 291 | 169 | 253 | 72.5 | 28.7 |
| T_{1/2} | hr | NA | NA | 5.89 | 5.89 | NA | NA |
| AUC ₗₐₛₜ | hr*ng/ml | 2199 | 3488 | 1941 | 2543 | 829 | 32.6 |
| AUC _{INF} | hr*ng/ml | NA | NA | 2120 | 2120 | NA | NA |
| MRT _{INF} | hr | NA | NA | 11.8 | 11.8 | NA | NA |
| Rsq_adjuseted | NA | NA | NA | 0.997 | NA | NA | NA |
| Regression Points | hr | NA | NA | 8~24 | NA | NA | NA |
| AUC ₗₐₛₜ | hr*μM | 2.78 | 4.40 | 2.45 | 3.21 | 1.05 | 32.6 |
| AUC _{INF} | hr*μM | NA | NA | 2.68 | 2.68 | NA | NA |
| Cₘₐₓ | μM | 0.376 | 0.367 | 0.213 | 0.319 | 0.0916 | 28.7 |

From the above results, it can be seen that under the same dosage, after using the technical scheme of Example 6, the concentration of dimyricetin-yl-diselenide in plasma was increased significantly, which indicates a promoting effect on bioavailability.

### Test Example 2

Pharmacodynamic study of subcutaneous transplantation in vivo

### (1) Cell Culture

Human liver cancer Hep3B cells were cultured in DMEM medium with 10% FBS in a 37° C. incubator containing 5% CO₂. Cells were cultured continuously for 5 generations and inoculated subcutaneously in mice. Mice was anesthetized with 3-4% isoflurane before vaccination. Serum-free medium containing 3.5×10⁶ Hep3B cells was subcutaneously injected into mice, with an injection volume of 100 μL.

### (2) Animal Grouping

When the tumor grows to an average of about 100-150 mm³, it is randomly divided into three groups based on tumor size and weight, with 8 mice in each group. The grouping situation is shown in the following table 4.

TABLE 4

| | | | Dosage | |
|---|---|---|---|---|
| Group | Test material | N* | (mg/kg) | Medication regimen |
| G1 | Solvent control group | 8 | N/A | Oral gavage once a |
| | | | | day for 4 weeks |
| G2 | Positive control drug | 8 | 2 mg/kg | Intraperitoneal |
| | Doxorubicin/doxorubicin | | | injection, twice a |
| | | | | week, for 4 weeks |
| G3 | DMS:PVP K30 = 1:4 | 8 | 8 mg/kg | Oral gavage once a |
| | (technical solution of | | | day for 4 weeks |
| | Example 6) | | | |

### (3) Observation

The appearance and behavior of each mouse were observed daily, form self-medication and continuously observed for 4 weeks. All abnormal appearance and behavioral activities were recorded in the clinical observation from Pengli Biological Laboratory. Testing drugs may have an impact on the digestive system of animals; if an animal experiences diarrhea, it should be recorded.

### (4) Measurement

Tumor volume: for subcutaneous transplant tumor models, tumor volume was measured twice a week for 4 consecutive weeks after grouping.

A calculation method for tumor volume (V) is as follows: V=(length×width²)/2.

A calculation method for relative tumor volume (RTV) of each nude mouse is: RTV=Vt/V0, where Vt is a measured volume per day and V0 is a volume at the beginning of treatment.
Animal weight: for subcutaneous transplant tumor models, the weight of mice was measured and recorded twice a week after grouping.
Food intake recording: after grouping, the remaining food weight in the rat cage was recorded twice a week to record the changes in food intake of each group of animals.
Animal state observation: the test drug may have an impact on the animal's digestive system; if an animal experiences diarrhea, it should be recorded.

### (5) Statistical Analysis

The results will be presented in the form of mean±S.E.M. A comparison between the two groups will be tested with Dunnett's multi comparison test. If p<0.05, it is considered that it is statistically significant.

### (6) Result Analysis:

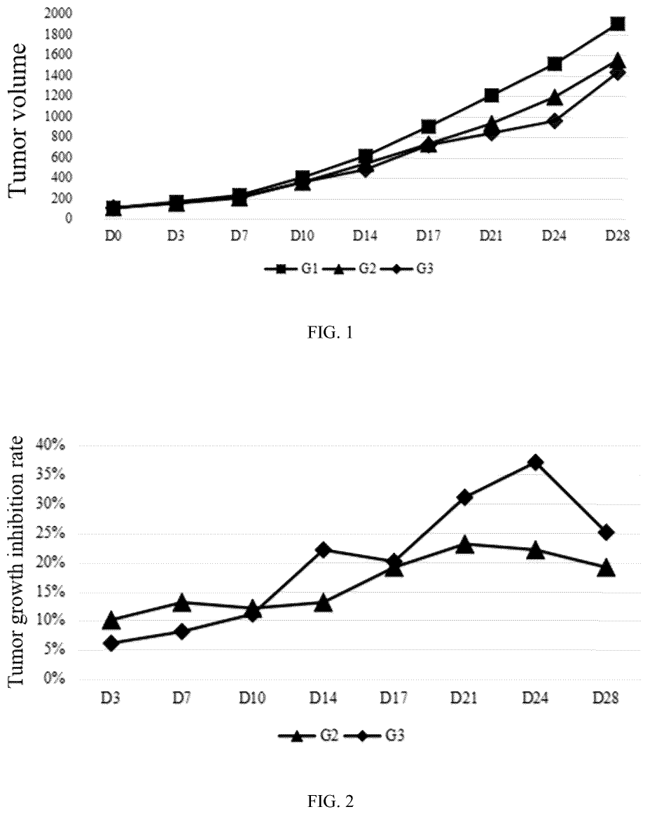

After administration, the measurement results of tumor volume in different mice are shown in the following table 5 and FIG. 1.

TABLE 5

| Group | D0 | D3 | D7 | D10 | D14 | D17 | D21 | D24 | D28 |
|---|---|---|---|---|---|---|---|---|---|
| G1 | 102.2 | 161.7 | 227.2 | 400.1 | 610.4 | 896.5 | 1201.4 | 1508.4 | 1899.05 |
| G2 | 102.7 | 146.9 | 199.6 | 352.6 | 534.6 | 727.1 | 927.2 | 1185.2 | 1545.98 |
| G3 | 102.1 | 151.4 | 209.2 | 353.9 | 477.4 | 715.8 | 833.1 | 951.5 | 1424.62 |

After administration, the tumor growth inhibition rates (TGI) of each group are shown in the following table 6 and FIG. 2.

TABLE 6

| Group | D3 | D7 | D10 | D14 | D17 | D21 | D24 | D28 |
|---|---|---|---|---|---|---|---|---|
| G2 | 10% | 13% | 12% | 13% | 19% | 23% | 22% | 19% |
| G3 | 6% | 8% | 11% | 22% | 20% | 31% | 37% | 25% |

The above results show that dimyricetin-yl-diselenide has a significant inhibitory effect on human liver cancer Hep3B cells in vivo, with an inhibition rate of up to 37%, and its inhibitory effect is better than that of the positive drug. This further proves the necessity of optimizing the formulation of the solid dispersion.

The above provides a detailed explanation of the embodiments of the present disclosure. However, the present disclosure is not limited to the above embodiments, and various changes can be made within the knowledge scope of those skilled in the art without departing from the purpose of the present disclosure.

What is claimed is:

1. A solid dispersion agent of dimyricetin-yl-diselenide, comprising dimyricetin-yl-diselenide and hydroxypropyl methylcellulose acetate succinate MG,
wherein a mass ratio of dimyricetin-yl-diselenide to hydroxypropyl methylcellulose acetate succinate MG is 1:3-5.

2. The solid dispersion agent of dimyricetin-yl-diselenide according to claim 1, wherein the mass ratio of dimyricetin-yl-diselenide to hydroxypropyl methylcellulose acetate succinate MG is 1:4.

3. A solid dispersion agent of dimyricetin-yl-diselenide, comprising dimyricetin-yl-diselenide and polyvinylpyrrolidone K30,
wherein a mass ratio of dimyricetin-yl-diselenide to polyvinylpyrrolidone K30 is 1:3-5.

4. The solid dispersion agent of dimyricetin-yl-diselenide according to claim 3, wherein the mass ratio of dimyricetin-yl-diselenide to polyvinylpyrrolidone K30 is 1:4.

5. An application of hydroxypropyl methylcellulose acetate succinate MG as a solid dispersion agent of dimyricetin-yl-diselenide.

6. The application according to claim 5, wherein a mass ratio of dimyricetin-yl-diselenide to hydroxypropyl methylcellulose acetate succinate MG is 1:3-5.

7. The application according to claim 6, wherein the mass ratio of dimyricetin-yl-diselenide to hydroxypropyl methylcellulose acetate succinate MG is 1:4.

8. An application of polyvinylpyrrolidone K30 as a solid dispersion agent of dimyricetin-yl-diselenide.

9. The application according to claim 8, wherein a mass ratio of dimyricetin-yl-diselenide to polyvinylpyrrolidone K30 is 1:3-5.

10. The application according to claim 9, wherein the mass ratio of dimyricetin-yl-diselenide to polyvinylpyrrolidone K30 is 1:4.
